(19) United States
(12) Patent Application Publication
(10) Pub. No.: US 2019/0194399 A1
(43) Pub. Date: Jun. 27, 2019

(54) Azeotropic or quasi-azeotropic composition comprising 1,1,1,2,2-pentafluoropropane and trans-1,3,3,3-tetrafluoropropene

(71) Applicant: Arkema France, Colombes (FR)

(72) Inventors: Dominque DEUR-BERT, Charly (FR); Wissam RACHED, Chaponost (FR); Laurent WENDLINGER, Soucieu en Jarrest (FR)

(73) Assignee: Arkema France, Colombes (FR)

(21) Appl. No.: 16/322,255

(22) PCT Filed: Aug. 8, 2017
(86) PCT No.: PCT/FR2017/052211

§ 371 Date: Jan. 31, 2019

(30) Foreign Application Priority Data
Aug. 10, 2016 (FR) 1657667
Feb. 1, 2017 (FR) 1750816

(51) Int. Cl. C08J 3/09; C09K 5/04; C09K 3/30; C08J 9/14; C07C 21/18; C07C 19/08; B01D 3/36; B01D 3/40; H01B 3/44

(52) U.S. Cl. CPC C08J 3/093; C09K 5/048; C09K 3/30; C08J 9/146; C07C 21/18; C07C 19/08; H01B 3/56; B01D 3/40; H01B 3/445; C08J 2203/162; C08J 2203/142; C08J 2203/182; B01D 3/36

(57) ABSTRACT

The present invention relates to compositions comprising 1,1,1,2,2-pentafluoropropane and trans-1,3,3,3-tetrafluoropropene, which are of use in numerous fields of application.

### TECHNICAL FIELD OF THE INVENTION

The present invention relates to compositions comprising 1,1,1,2,2-pentafluoropropane and trans-1,3,3,3-tetrafluoropropene, which are of use in numerous fields of application.

Fluids based on halocarbons have found numerous applications in varied industrial fields, in particular as heat-transfer fluids, propellants, foaming agents, blowing agents, gaseous dielectrics, monomer or polymerization medium, support fluids, agents for abrasives, drying agents and fluids for energy production units. Particular importance is given to fluids having a low impact on the environment, such as, for example, 2,3,3,3-tetrafluoropropene.

Certain processes for the production of 2,3,3,3-tetrafluoropropene involve the formation of 1,1,1,2,2-pentafluoropropane and of different isomers, such as trans-1,3,3,3-tetrafluoropropene. Thus, the final purification of 2,3,3,3-tetrafluoropropene requires the separation of the latter from 1,1,1,2,2-pentafluoropropane and from trans-1,3,3,3-tetrafluoropropene, which are the main impurities. 1,1,1,2,2-Pentafluoropropane is a compound which can be recycled in the process for the production of 2,3,3,3-tetrafluoropropene in order to promote the reaction. Contrariwise, the trans-1,3,3,3-tetrafluoropropene has to be removed from the reaction loop in order to limit the formation of isomers. It is thus necessary to separate the 1,1,1,2,2-pentafluoropropane from the trans-1,3,3,3-tetrafluoropropene in order to be able to recycle the 1,1,1,2,2-pentafluoropropane essentially devoid of trans-1,3,3,3-tetrafluoropropene.

1,1,1,2,2-Pentafluoropropane and trans-1,3,3,3-tetrafluoropropene cannot be separated by conventional distillation. The separation of 1,1,1,2,2-pentafluoropropane from trans-1,3,3,3-tetrafluoropropene by extractive distillation is known from FR 15/63163, FR 15/63169 and FR 15/63168.

There thus exists a need for effective methods for the separation of 1,1,1,2,2-pentafluoropropane and trans-1,3,3,3-tetrafluoropropene.

### SUMMARY OF THE INVENTION

According to a first aspect, the present invention relates to an azeotropic or quasi-azeotropic composition comprising 1,1,1,2,2-pentafluoropropane and trans-1,3,3,3-tetrafluoropropene.

According to a preferred embodiment, the composition comprises from 0.01 mol % to 99.99 mol % of trans-1,3,3,3-tetrafluoropropene and from 0.01 mol % to 99.99 mol % of 1,1,1,2,2-pentafluoropropane, based on the total amount in moles of the composition, advantageously from 51 mol % to 99 mol % of trans-1,3,3,3-tetrafluoropropene and from 1 mol % to 49 mol % of 1,1,1,2,2-pentafluoropropane, based on the total amount in moles of the composition, in particular from 60 mol % to 99 mol % of trans-1,3,3,3-tetrafluoropropene and from 1 mol % to 40 mol % of 1,1,1,2,2-pentafluoropropane, based on the total amount in moles of the composition.

The boiling point of the composition can be between −50° C. and 100° C., advantageously between −40° C. and 80° C., preferably between −35° C. and 70° C., in particular between −30° C. and 60° C., more particularly from −30° C. to 20° C., favourably from −30° C. to 10° C., more favourably from −30° C. to 0° C., particularly favourably from −30° C. to −5° C. The boiling point can also be between −50° C. and 0° C., advantageously between −40° C. and −5° C., preferably between −35° C. and −10° C.

Preferably, the pressure is between 0.2 and 30 bara, preferably between 0.3 and 20 bara, in particular between 0.5 and 16 bara, more particularly between 0.6 and 13 bara.

According to a specific embodiment, the composition comprises from 80 mol % to 99 mol % of trans-1,3,3,3-tetrafluoropropene and from 1 mol % to 20 mol % of 1,1,1,2,2-pentafluoropropane, advantageously from 85 mol % to 97 mol % of trans-1,3,3,3-tetrafluoropropene and from 3 mol % to 15 mol % of 1,1,1,2,2-pentafluoropropane, preferably from 88 mol % to 95 mol % of trans-1,3,3,3-tetrafluoropropene and from 5 mol % to 12 mol % of 1,1,1,2,2-pentafluoropropane, based on the total amount in moles of the composition. Preferably, the composition is azeotropic.

According to a specific embodiment, the composition according to the present invention as described above consists of 1,1,1,2,2-pentafluoropropane and trans-1,3,3,3-tetrafluoropropene.

According to a second aspect, the present invention provides a process for the separation of the composition according to the present invention, characterized in that it comprises a stage of extractive distillation of the said composition in the presence of a solvent, in order to obtain a first stream predominantly comprising 1,1,1,2,2-pentafluoropropane and a second stream predominantly comprising trans-1,3,3,3-tetrafluoropropene. The expression “a first stream predominantly comprising 1,1,1,2,2-pentafluoropropane” means that the first stream comprises an amount in moles of 1,1,1,2,2-pentafluoropropane which is greater than the amount in moles of trans-1,3,3,3-tetrafluoropropene. The expression “a second stream predominantly comprising trans-1,3,3,3-tetrafluoropropene” means that the second stream comprises an amount in moles of trans-1,3,3,3-tetrafluoropropene which is greater than the amount in moles of 1,1,1,2,2-pentafluoropropane.

### BRIEF DESCRIPTION OF THE FIGURES

FIG. 1 represents a graph illustrating the relative volatility of a composition of 1,1,1,2,2-pentafluoropropane and of trans-1,3,3,3-tetrafluoropropene as a function of their respective molar fractions at T=50° C.

FIG. 2 represents a graph illustrating the relative volatility of a composition of 1,1,1,2,2-pentafluoropropane and of trans-1,3,3,3-tetrafluoropropene as a function of their respective molar fractions at T=−30° C.

### DETAILED DESCRIPTION OF THE PRESENT INVENTION

The expression “quasi-azeotropic” has a broad meaning and is intended to include compositions which are strictly azeotropic and those which behave like an azeotropic mixture.

The volatility of a compound A is represented by the ratio of the molar fraction in the gas phase (y_{A}) to the molar fraction in the liquid phase (x_{A}) under equilibrium conditions (at pressure and temperature equilibrium): α=y_{A}/x_{A}. The volatility of a compound B is represented by the ratio of the molar fraction in the gas phase (y_{B}) to the molar fraction in the liquid phase (x_{B}) under equilibrium conditions (at pressure and temperature equilibrium): α=y_{B}/x_{B}. The relative volatility makes it possible to measure the ease of separation of two compounds A and B. It is the ratio of the volatilities of the 2 compounds: α_{A,B}=y_{A}x_{B}=y_{B}/x_{A}. The greater the volatility, the more the mixture can be easily separated.

When the relative volatility is equal to 1 or between 0.95 and 1.05, this means that the mixture is azeotropic. When the relative volatility is between 0.85 and 1.15, this means that the mixture is quasi-azeotropic.

A subject-matter of the present invention is an azeotropic or quasi-azeotropic composition comprising the compounds 1,1,1,2,2-pentafluoropropane and trans-1,3,3,3-tetrafluoropropene.

In that which follows, the following compounds represent:
1,1,1,2,2-pentafluoropropane: HFC-245cb
trans-1,3,3,3-tetrafluoropropene: HFO-1234zeE

According to a preferred embodiment, the composition comprises from 0.01 mol % to 99.99 mol % of trans-1,3,3,3-tetrafluoropropene and from 0.01 mol % to 99.99 mol % of 1,1,1,2,2-pentafluoropropane, based on the total amount in moles of the composition.

More preferably, the said composition comprises from 0.01 mol % to 99.99 mol % of trans-1,3,3,3-tetrafluoropropene, advantageously the said composition comprises from 0.5 mol % to 99.5 mol % of trans-1,3,3,3-tetrafluoropropene, preferably the said composition comprises from 10 mol % to 99 mol % of trans-1,3,3,3-tetrafluoropropene, more preferably the said composition comprises from 20 mol % to 99 mol % of trans-1,3,3,3-tetrafluoropropene, in particular the said composition comprises from 30 mol % to 99 mol % of trans-1,3,3,3-tetrafluoropropene, more particularly the said composition comprises from 40 mol % to 99 mol % of trans-1,3,3,3-tetrafluoropropene, favourably the said composition comprises from 50 mol % to 99 mol % of trans-1,3,3,3-tetrafluoropropene, more favourably the said composition comprises from 60 mol % to 99 mol % of trans-1,3,3,3-tetrafluoropropene, more favourably still the said composition comprises from 70 mol % to 99 mol % of trans-1,3,3,3-tetrafluoropropene, on the basis of the total amount in moles of the composition.

Preferably, the said composition comprises from 0.01 mol % to 99.9 mol % of 1,1,1,2,2-pentafluoropropane, advantageously the said composition comprises from 0.5 mol % to 99.5 mol % of 1,1,1,2,2-pentafluoropropane, preferably the said composition comprises from 1 mol % to 90 mol % of 1,1,1,2,2-pentafluoropropane, more preferably the said composition comprises from 1 mol % to 80 mol % of 1,1,1,2,2-pentafluoropropane, in particular the said composition comprises from 1 mol % to 70 mol % of 1,1,1,2,2-pentafluoropropane, more particularly the said composition comprises from 1 mol % to 60 mol % of 1,1,1,2,2-pentafluoropropane, favourably the said composition comprises from 1 mol % to 50 mol % of 1,1,1,2,2-pentafluoropropane, more favourably the said composition comprises from 1 mol % to 40 mol % of 1,1,1,2,2-pentafluoropropane, more favourably still the said composition comprises from 1 mol % to 30 mol % of 1,1,1,2,2-pentafluoropropane, on the basis of the total amount in moles of the composition.

According to a preferred embodiment, the composition comprises from 0.01 mol % to 99.99 mol % of trans-1,3,3,3-tetrafluoropropene, advantageously from 0.5 mol % to 99.5 mol %, preferably from 10 mol % to 99 mol %, more preferably from 20 mol % to 99 mol %, in particular from 30 mol % to 99 mol %, more particularly from 40 mol % to 99 mol %, favourably from 50 mol % to 99 mol %, more favourably from 60 mol % to 99 mol %, more favourably still from 70 mol % to 99 mol % of trans-1,3,3,3-tetrafluoropropene; and from 0.01 mol % to 99.9 mol % of 1,1,1,2,2-pentafluoropropane, advantageously from 0.5 mol % to 99.5 mol %, preferably from 1 mol % to 90 mol %, more preferably from 1 mol % to 80 mol %, in particular from 1 mol % to 70 mol %, more particularly from 1 mol % to 60 mol %, favourably from 1 mol % to 50 mol %, more favourably from 1 mol % to 40 mol %, more favourably still from 1 mol % to 30 mol % of 1,1,1,2,2-pentafluoropropane, based on the total amount in moles of the composition.

The boiling point of the composition is between −50° C. and 100° C., advantageously between −40° C. and 80° C., preferably between −35° C. and 70° C., in particular between −30° C. and 60° C., more particularly from −30° C. to 20° C., favourably from −30° C. to 10° C., more favourably from −30° C. to 0° C., particularly favourably from −30° C. to −5° C.

According to a preferred embodiment, the composition comprises from 0.01 mol % to 99.99 mol % of trans-1,3,3,3-tetrafluoropropene, advantageously from 0.5 mol % to 99.5 mol %, preferably from 10 mol % to 99 mol %, more preferably from 20 mol % to 99 mol %, in particular from 30 mol % to 99 mol %, more particularly from 40 mol % to 99 mol %, favourably from 50 mol % to 99 mol %, more favourably from 60 mol % to 99 mol %, more favourably still from 70 mol % to 99 mol % of trans-1,3,3,3-tetrafluoropropene; and from 0.01 mol % to 99.9 mol % of 1,1,1,2,2-pentafluoropropane, advantageously from 0.5 mol % to 99.5 mol %, preferably from 1 mol % to 90 mol %, more preferably from 1 mol % to 80 mol %, in particular from 1 mol % to 70 mol %, more particularly from 1 mol % to 60 mol %, favourably from 1 mol % to 50 mol %, more favourably from 1 mol % to 40 mol %, more favourably still from 1 mol % to 30 mol % of 1,1,1,2,2-pentafluoropropane, based on the total amount in moles of the composition, and the said composition has a boiling point of between −50° C. and 100° C., advantageously between −40° C. and 80° C., preferably between −35° C. and 70° C., in particular between −30° C. and 60° C., more particularly from −30° C. to 20° C., favourably from −30° C. to 10° C., more favourably from −30° C. to 0° C., particularly favourably from −30° C. to −5° C.

Preferably, the pressure is between 0.2 and 30 bara, preferably between 0.3 and 20 bara, in particular between 0.5 and 16 bara, more particularly between 0.6 and 13 bara.

Preferably, the composition comprises from 0.01 mol % to 99.99 mol % of trans-1,3,3,3-tetrafluoropropene, advantageously from 0.5 mol % to 99.5 mol %, preferably from 10 mol % to 99 mol %, more preferably from 20 mol % to 99 mol %, in particular from 30 mol % to 99 mol %, more particularly from 40 mol % to 99 mol %, favourably from 50 mol % to 99 mol %, more favourably from 60 mol % to 99 mol %, more favourably still from 70 mol % to 99 mol % of trans-1,3,3,3-tetrafluoropropene; and from 0.01 mol % to 99.9 mol % of 1,1,1,2,2-pentafluoropropane, advantageously from 0.5 mol % to 99.5 mol %, preferably from 1 mol % to 90 mol %, more preferably from 1 mol % to 80 mol %, in particular from 1 mol % to 70 mol %, more particularly from 1 mol % to 60 mol %, favourably from 1 mol % to 50 mol %, more favourably from 1 mol % to 40 mol %, more favourably still from 1 mol % to 30 mol % of 1,1,1,2,2-pentafluoropropane, based on the total amount in moles of the composition, and the said composition has a boiling point of between −50° C. and 100° C., advantageously between −40° C. and 80° C., preferably between −35° C. and 70° C., in particular between −30° C. and 60° C., more particularly from −30° C. to 20° C., favourably from −30° C. to 10° C., more favourably from −30° C. to 0° C., particularly favourably from −30° C. to −5° C., at a pressure of between 0.2 and 30 bara, preferably between 0.3 and 20 bara, in particular between 0.5 and 16 bara, more particularly between 0.6 and 13 bara.

The boiling point can also be between −50° C. and 0° C., advantageously between −40° C. and −5° C., preferably between −35° C. and −10° C.

Thus, the composition comprises from 0.01 mol % to 99.99 mol % of trans-1,3,3,3-tetrafluoropropene, advantageously from 0.5 mol % to 99.5 mol %, preferably from 10 mol % to 99 mol %, more preferably from 20 mol % to 99 mol %, in particular from 30 mol % to 99 mol %, more particularly from 40 mol % to 99 mol %, favourably from 50 mol % to 99 mol %, more favourably from 60 mol % to 99 mol %, more favourably still from 70 mol % to 99 mol % of trans-1,3,3,3-tetrafluoropropene; and from 0.01 mol % to 99.9 mol % of 1,1,1,2,2-pentafluoropropane, advantageously from 0.5 mol % to 99.5 mol %, preferably from 1 mol % to 90 mol %, more preferably from 1 mol % to 80 mol %, in particular from 1 mol % to 70 mol %, more particularly from 1 mol % to 60 mol %, favourably from 1 mol % to 50 mol %, more favourably from 1 mol % to 40 mol %, more favourably still from 1 mol % to 30 mol % of 1,1,1,2,2-pentafluoropropane, based on the total amount in moles of the composition, and the said composition has a boiling point of between −50° C. and 0° C., advantageously between −40° C. and −5° C., preferably between −35° C. and −10° C.

Preferably, the composition comprises from 0.01 mol % to 99.99 mol % of trans-1,3,3,3-tetrafluoropropene, advantageously from 0.5 mol % to 99.5 mol %, preferably from 10 mol % to 99 mol %, more preferably from 20 mol % to 99 mol %, in particular from 30 mol % to 99 mol %, more particularly from 40 mol % to 99 mol %, favourably from 50 mol % to 99 mol %, more favourably from 60 mol % to 99 mol %, more favourably still from 70 mol % to 99 mol % of trans-1,3,3,3-tetrafluoropropene; and from 0.01 mol % to 99.9 mol % of 1,1,1,2,2-pentafluoropropane, advantageously from 0.5 mol % to 99.5 mol %, preferably from 1 mol % to 90 mol %, more preferably from 1 mol % to 80 mol %, in particular from 1 mol % to 70 mol %, more particularly from 1 mol % to 60 mol %, favourably from 1 mol % to 50 mol %, more favourably from 1 mol % to 40 mol %, more favourably still from 1 mol % to 30 mol % of 1,1,1,2,2-pentafluoropropane, based on the total amount in moles of the composition, and the said composition has a boiling point of between −50° C. and 0° C., advantageously between −40° C. and −5° C., preferably between −35° C. and −10° C., at a pressure of between 0.2 and 30 bara, preferably between 0.3 and 20 bara, in particular between 0.5 and 16 bara, more particularly between 0.6 and 13 bara.

According to a specific embodiment, the composition is azeotropic.

Preferably, the said azeotropic composition can comprise from 81 mol % to 99 mol % of trans-1,3,3,3-tetrafluoropropene, advantageously the said azeotropic composition can comprise from 82 mol % to 98 mol % of trans-1,3,3,3-tetrafluoropropene, preferably the said azeotropic composition can comprise from 83 mol % to 98 mol % of trans-1,3,3,3-tetrafluoropropene, more preferably the said azeotropic composition can comprise from 84 mol % to 97 mol % of trans-1,3,3,3-tetrafluoropropene, in particular the said azeotropic composition can comprise from 85 mol % to 97 mol % of trans-1,3,3,3-tetrafluoropropene, more particularly the said azeotropic composition can comprise from 86 mol % to 96 mol % of trans-1,3,3,3-tetrafluoropropene, favourably the said azeotropic composition can comprise from 87 mol % to 96 mol % of trans-1,3,3,3-tetrafluoropropene, more favourably the said azeotropic composition can comprise from 88 mol % to 95 mol % of trans-1,3,3,3-tetrafluoropropene, particularly favourably from 88 mol % to 94 mol % of trans-1,3,3,3-tetrafluoropropene, more particularly favourably from 88 mol % to 93 mol % of trans-1,3,3,3-tetrafluoropropene, on the basis of the total amount in moles of the azeotropic composition.

Preferably, the said azeotropic composition can comprise from 1 mol % to 19 mol % of 1,1,1,2,2-pentafluoropropane, advantageously the said azeotropic composition can comprise from 2 mol % to 18 mol % of 1,1,1,2,2-pentafluoropropane, preferably the said azeotropic composition can comprise from 2 mol % to 17 mol % of 1,1,1,2,2-pentafluoropropane, more preferably the said azeotropic composition can comprise from 3 mol % to 16 mol % of 1,1,1,2,2-pentafluoropropane, in particular the said azeotropic composition can comprise from 3 mol % to 15 mol % of 1,1,1,2,2-pentafluoropropane, more particularly the said azeotropic composition can comprise from 4 mol % to 14 mol % of 1,1,1,2,2-pentafluoropropane, favourably the said azeotropic composition can comprise from 4 mol % to 13 mol % of 1,1,1,2,2-pentafluoropropane, more favourably the said azeotropic composition can comprise from 5 mol % to 12 mol % of 1,1,1,2,2-pentafluoropropane, particularly favourably from 6 mol % to 12 mol % of 1,1,1,2,2-pentafluoropropane, more particularly favourably from 7 mol % to 12 mol % of 1,1,1,2,2-pentafluoropropane, on the basis of the total amount in moles of the azeotropic composition.

In particular, the said azeotropic composition can comprise from 81 mol % to 99 mol % of trans-1,3,3,3-tetrafluoropropene, advantageously from 82 mol % to 98 mol %, preferably from 83 mol % to 98 mol %, more preferably from 84 mol % to 97 mol %, in particular from 85 mol % to 97 mol %, more particularly from 86 mol % to 96 mol %, favourably from 87 mol % to 96 mol %, more favourably from 88 mol % to 95 mol % of trans-1,3,3,3-tetrafluoropropene, particularly favourably from 88 mol % to 94 mol % of trans-1,3,3,3-tetrafluoropropene, more particularly favourably from 88 mol % to 93 mol % de trans-1,3,3,3-tetrafluoropropene; and from 1 mol % to 19 mol % of 1,1,1,2,2-pentafluoropropane, advantageously from 2 mol % to 18 mol % of 1,1,1,2,2-pentafluoropropane, preferably from 2 mol % to 17 mol %, more preferably from 3 mol % to 16 mol %, in particular from 3 mol % to 15 mol %, more particularly from 4 mol % to 14 mol %, favourably from 4 mol % to 13 mol %, more favourably from 5 mol % to 12 mol %, particularly favourably from 6 mol % to 12 mol % of 1,1,1,2,2-pentafluoropropane, more particularly favourably from 7 mol % to 12 mol % of 1,1,1,2,2-pentafluoropropane, based on the total amount in moles of the azeotropic composition.

The said azeotropic composition can have a boiling point of between −50° C. and 100° C., advantageously between −40° C. and 80° C., preferably between −35° C. and 70° C., in particular between −30° C. and 60° C., more particularly from −30° C. to 20° C., favourably from −30° C. to 10° C., more favourably from −30° C. to 0° C., particularly favourably from −30° C. to −5° C. The said azeotropic composition can also have a boiling point of between −50° C. and 0° C., advantageously between −40° C. and −5° C., preferably between −35° C. and −10° C., more preferably from −35° C. to −20° C., in particular from −35° C. to −25° C. Alternatively, the said azeotropic composition can have a boiling point of between 40° C. and 60° C., preferably between 45° C. and 55° C.

Preferably, the said azeotropic composition can comprise from 81 mol % to 99 mol % of trans-1,3,3,3-tetrafluoropropene, advantageously from 82 mol % to 98 mol %, preferably from 83 mol % to 98 mol %, more preferably from 84 mol % to 97 mol %, in particular from 85 mol % to 97 mol %, more particularly from 86 mol % to 96 mol %, favourably from 87 mol % to 96 mol %, more favourably from 88 mol % to 95 mol % of trans-1,3,3,3-tetrafluoropropene; and from 1 mol % to 19 mol % of 1,1,1,2,2-pentafluoropropane, advantageously from 2 mol % to 18 mol % of 1,1,1,2,2-pentafluoropropane, preferably from 2 mol % to 17 mol %, more preferably from 3 mol % to 16 mol %, in particular from 3 mol % to 15 mol %, more particularly from 4 mol % to 14 mol %, favourably from 4 mol % to 13 mol %, more favourably from 5 mol % to 12 mol %, based on the total amount in moles of the azeotropic composition; and the said azeotropic composition has a boiling point of between −50° C. and 100° C., advantageously between −40° C. and 80° C., preferably between −35° C. and 70° C., in particular between −30° C. and 60° C., more particularly from −30° C. to 20° C., favourably from −30° C. to 10° C., more favourably from −30° C. to 0° C., particularly favourably from −30° C. to −5° C.

Preferably, the said azeotropic composition can comprise from 81 mol % to 99 mol % of trans-1,3,3,3-tetrafluoropropene, advantageously from 82 mol % to 98 mol %, preferably from 83 mol % to 98 mol %, more preferably from 84 mol % to 97 mol %, in particular from 85 mol % to 97 mol %, more particularly from 86 mol % to 96 mol %, favourably from 87 mol % to 96 mol %, more favourably from 88 mol % to 95 mol % of trans-1,3,3,3-tetrafluoropropene; and from 1 mol % to 19 mol % of 1,1,1,2,2-pentafluoropropane, advantageously from 2 mol % to 18 mol % of 1,1,1,2,2-pentafluoropropane, preferably from 2 mol % to 17 mol %, more preferably from 3 mol % to 16 mol %, in particular from 3 mol % to 15 mol %, more particularly from 4 mol % to 14 mol %, favourably from 4 mol % to 13 mol %, more favourably from 5 mol % to 12 mol %, based on the total amount in moles of the azeotropic composition; and the said azeotropic composition has a boiling point of between −50° C. and 0° C., advantageously between −40° C. and −5° C., preferably between −35° C. and −10° C., more preferably from −35° C. to −20° C., in particular from −35° C. to −25° C. Alternatively, the said azeotropic composition can have a boiling point of between 40° C. and 60° C., preferably between 45° C. and 55° C.

According to a preferred embodiment, the pressure can be between 0.2 and 30 bara, preferably between 0.3 and 20 bara, in particular between 0.5 and 16 bara, more particularly between 0.6 and 13 bara.

Preferably, the said azeotropic composition can comprise from 81 mol % to 99 mol % of trans-1,3,3,3-tetrafluoropropene, advantageously from 82 mol % to 98 mol %, preferably from 83 mol % to 98 mol %, more preferably from 84 mol % to 97 mol %, in particular from 85 mol % to 97 mol %, more particularly from 86 mol % to 96 mol %, favourably from 87 mol % to 96 mol %, more favourably from 88 mol % to 95 mol % of trans-1,3,3,3-tetrafluoropropene; and from 1 mol % to 19 mol % of 1,1,1,2,2-pentafluoropropane, advantageously from 2 mol % to 18 mol % of 1,1,1,2,2-pentafluoropropane, preferably from 2 mol % to 17 mol %, more preferably from 3 mol % to 16 mol %, in particular from 3 mol % to 15 mol %, more particularly from 4 mol % to 14 mol %, favourably from 4 mol % to 13 mol %, more favourably from 5 mol % to 12 mol %, based on the total amount in moles of the azeotropic composition; and has a boiling point of between −50° C. and 100° C., advantageously between −40° C. and 80° C., preferably between −35° C. and 70° C., in particular between −30° C. and 60° C., more particularly from −30° C. to 20° C., favourably from −30° C. to 10° C., more favourably from −30° C. to 0° C., particularly favourably from −30° C. to −5° C., at a pressure of between 0.2 and 30 bara, preferably between 0.3 and 20 bara, in particular between 0.5 and 16 bara, more particularly between 0.6 and 13 bara.

Preferably, the said azeotropic composition can comprise from 81 mol % to 99 mol % of trans-1,3,3,3-tetrafluoropropene, advantageously from 82 mol % to 98 mol %, preferably from 83 mol % to 98 mol %, more preferably from 84 mol % to 97 mol %, in particular from 85 mol % to 97 mol %, more particularly from 86 mol % to 96 mol %, favourably from 87 mol % to 96 mol %, more favourably from 88 mol % to 95 mol % of trans-1,3,3,3-tetrafluoropropene; and from 1 mol % to 19 mol % of 1,1,1,2,2-pentafluoropropane, advantageously from 2 mol % to 18 mol % of 1,1,1,2,2-pentafluoropropane, preferably from 2 mol % to 17 mol %, more preferably from 3 mol % to 16 mol %, in particular from 3 mol % to 15 mol %, more particularly from 4 mol % to 14 mol %, favourably from 4 mol % to 13 mol %, more favourably from 5 mol % to 12 mol %, based on the total amount in moles of the azeotropic composition; and the said azeotropic composition has a boiling point of between −50° C. and 0° C., advantageously between −40° C. and −5° C., preferably between −35° C. and −10° C., at a pressure of between 0.2 and 30 bara, preferably between 0.3 and 20 bara, in particular between 0.5 and 16 bara, more particularly between 0.6 and 13 bara. In particular, the said azeotropic or quasi-azeotropic composition as described above consists of 1,1,1,2,2-pentafluoropropane and trans-1,3,3,3-tetrafluoropropene.

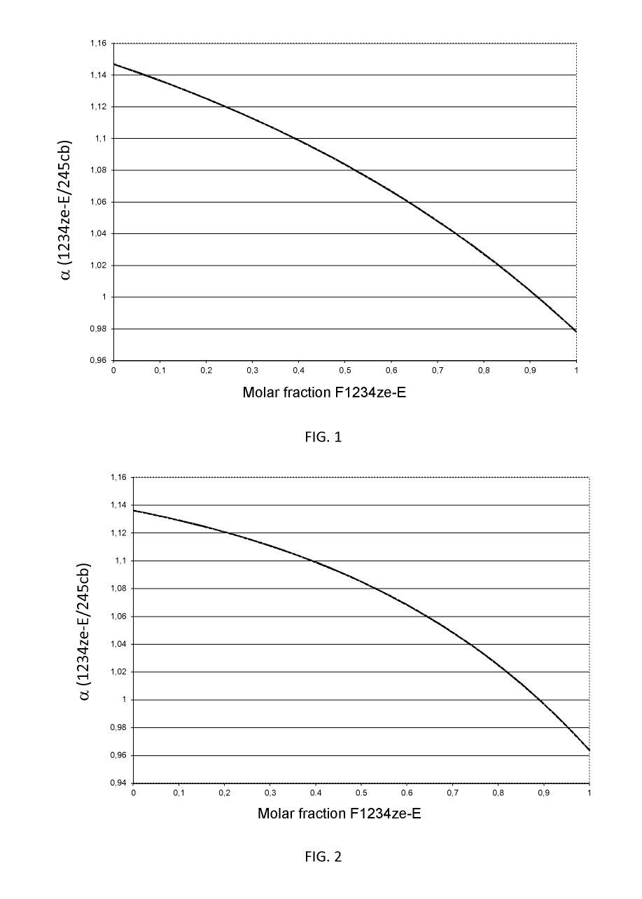

The relative volatilities of the compositions according to the present invention are represented in FIGS. 1 and 2. FIG. 1 more particularly illustrates the relative volatility of a composition of 1,1,1,2,2-pentafluoropropane and of trans-1,3,3,3-tetrafluoropropene as a function of their respective molar fractions at a temperature of 50° C. FIG. 2 more particularly illustrates the relative volatility of a composition of 1,1,1,2,2-pentafluoropropane and of trans-1,3,3,3-tetrafluoropropene as a function of their respective molar fractions at −30° C.

According to another aspect of the present invention, a process for the separation of 1,1,1,2,2-pentafluoropropane and of trans-1,3,3,3-tetrafluoropropene is provided. The said process comprises a stage of extractive distillation of an azeotropic or quasi-azeotropic composition as described above in the presence of an organic solvent in order to obtain a first stream predominantly or essentially comprising 1,1,1,2,2-pentafluoropropane and a second stream predominantly or essentially comprising trans-1,3,3,3-tetrafluoropropene.

Preferably, the process for the separation of any one of the compositions described above comprises a stage of extractive distillation of the said composition in the presence of an organic solvent, in order to obtain a first stream predominantly comprising 1,1,1,2,2-pentafluoropropane and a second stream predominantly comprising trans-1,3,3,3-tetrafluoropropene; preferably, the organic solvent is present in the second stream; in particular, the organic solvent is selected from the group consisting of ethylamine, isopropylamine, n-propylamine, diethylamine, propanone, tetrahydrofuran, ethyl acetate, butanone, 3-pentylamine, 2-methoxyethanamine, 1,4-dioxane, 3-pentanone, 2-pentanone, n-pentylamine, 1,3-dioxane, 1,2-diaminoethane, 1,2-propanediamine, 2-methoxyethanol, n-butyl acetate and 1-ethoxy-2-propanol.

1. An azeotropic or quasi-azeotropic composition comprising from 51 mol % to 99 mol % of trans-1,3,3,3-tetrafluoropropene and from 1 mol % to 49 mol % of 1,1,1,2,2-pentafluoropropane, based on the total amount in moles of the composition, the said composition having a boiling point of between −50° C. and 0° C.

2. The composition according to claim 1, wherein the composition comprises from 60 mol % to 99 mol % of trans-1,3,3,3-tetrafluoropropene and from 1 mol % to 40 mol % of 1,1,1,2,2-pentafluoropropane, based on the total amount in moles of the composition.

3. The composition according to claim 1, wherein the boiling point is between −40° C. and −5° C.

4. The composition according to claim 1, wherein the pressure is between 0.2 and 30 bara.

5. The composition according to claim 1, wherein the composition comprises from 80 mol % to 99 mol % of trans-1,3,3,3-tetrafluoropropene and from 1 mol % to 20 mol % of 1,1,1,2,2-pentafluoropropane, based on the total amount in moles of the composition.

6. The composition according to claim 1, wherein the composition is azeotropic.

7. The composition according to claim 1, wherein the composition consists of 1,1,1,2,2-pentafluoropropane and trans-1,3,3,3-tetrafluoropropene.

8. A for the separation of the composition according to claim 1, wherein the process comprises a stage of extractive distillation of the said composition in the presence of an organic solvent, in order to obtain a first stream predominantly comprising 1,1,1,2,2-pentafluoropropane and a second stream predominantly comprising trans-1,3,3,3-tetrafluoropropene.
